(19) United States
(12) Patent Application Publication
(10) Pub. No.: US 2022/0362417 A1
(43) Pub. Date: Nov. 17, 2022

(54) Ultraviolet Rays Radiating Personal Protective Eyewear Device

(71) Applicant: Robert Barnes, Fort Walton Beach, FL (US)

(72) Inventor: Robert Barnes, Fort Walton Beach, FL (US)

(21) Appl. No.: 17/711,628

(22) Filed: Apr. 1, 2022

Related U.S. Application Data
(60) Provisional application No. 63/188,662, filed on May 14, 2021.

(51) Int. Cl. A61L 2/10; G02C 11/04; G02C 11/00

(52) U.S. Cl. CPC A61L 2/10; G02C 11/04; G02C 11/10; A61L 2202/11

(57) ABSTRACT

The present invention relates to a personal protective eyewear device. The eyewear/eyeglass device is configured to function as personal protective equipment for virtually anyone while in high-traffic public areas like retail stores, schools, offices, etc. The eyewear device comprises a plurality of LED wafers disposed at the bottom of the frame, or on a bracket that is attached to the bottom of the frame, for emitting and projecting far-UVC light in the range 207-222 nm to create a shield for pathogens. The shield disinfects both the inhaled and exhaled air.

### CROSS-REFERENCE TO RELATED APPLICATION

The present application claims priority to, and the benefit of U.S. Provisional Application No. 63/188,662, which was filed on May 14, 2021 and is incorporated herein by reference in its entirety.

### FIELD OF THE INVENTION

The present invention relates generally to the field of eyewear. More specifically, the present invention relates to eyeglasses which project ultraviolet rays to create a protective shield against airborne pathogens and other microbes for the user. The eyewear device comprises a plurality of LED wafers for projecting far-UVC lights that are safe for humans and also effective in killing viruses and other pathogens. The LED wafers are powered by a rechargeable battery and can be activated by a ON/OFF button. More specifically, the eyewear device can be used as personal protective equipment for virtually anyone while in high-traffic public areas, like retail stores, schools, offices and more. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

There has been great interest in a variety of ways to effectively reduce the risk of infection from pathogens and other air borne microbes not only in enclosed spaces, hospitals, hospices, non-healthcare settings but also in other public places where people congregate. Both healthcare and non-healthcare environments contain a diverse population of microorganisms that can cause infection. A person with a communicable disease can infect a multitude of people when they come in close contact with the infected person.

By way of background, people use a variety of personal protective equipment (PPE) for protecting themselves from pathogens. These PPEs include, for example, a facemask, a face shield, gloves etc. Further, people are in danger of inhaling pathogens when they are in a crowded place, which has become more prevalent during pandemics, such as during the Covid-19 pandemic.

Generally, wearing a facemask is a non-pharmaceutical method for reducing the risk of respiratory infection and inhaling pathogens such as bacteria, SARS, etc. Examples of common facemasks include disposable surgical facemasks, fabric facemasks and N95 respirators. These facemasks reduce transmission of airborne pathogens by preventing a user from directly touching his or her nose and mouth with dirty hands and/or by containing large liquid droplets expelled during sneezing or coughing. However, these facemasks are generally unable to disinfect the air being inhaled or exhaled, and sometimes cannot block airborne viruses, most of which are smaller than 0.3 microns. Users also find it difficult and uncomfortable to wear such facemasks, which makes them reluctant to wear facemasks, especially in public places. Furthermore, if the masks are not form-fitted, a significant amount of air can leak through the periphery of the mask, greatly reducing the mask's effectiveness. Facemasks also affect the efficiency of communication between people. Further, facemasks also lead to other inconveniences, such as fogging of lenses in cold weather for eye-glass wearers from leakage of moist air. Similarly, face shields can also allow pathogens to leak through from the sides, and people generally find wearing face shields suffocating and uncomfortable.

Both facemask and face shields also limit the breathing ability of a user and a user with a breathing disease such as Asthma cannot wear a facemask for a long time. Similarly, users with other diseases may find it difficult to wear masks and shields even for a short duration.

Gloves are useful in protecting hands from touching contaminated surfaces, however gloves are not effective in protection from inhaling contaminated air. Further, air purifier devices can be used in enclosed and smaller places, but are not portable and cannot be used in public places.

Therefore, users, medical agencies and even regulatory authorities desire a more effective personal protective device for protection from inhaling and exchanging pathogens and other microbes that can be used by everyone in all situations.

Therefore, there exists a long felt need in the art for an improved personal protective device that provides a shield to users to protect against the spread of germs, viruses and bacteria. There is also a long felt need in the art for an improved personal protective device that is both comfortable to the user and effective in protection from pathogens. Additionally, there is a long felt need in the art for an improved personal protective device that can be used by anyone including elderly, asthmatic and sick users. Moreover, there is a long felt need in the art for an improved personal protective device that may be in a different form than facemasks and face shields. Further, there is a long felt need in the art for an improved personal protective device that can be used in high-traffic public areas like retail stores, schools, offices, etc. Finally, there is a long felt need in the art for an improved personal protective device that provides users with a way to maintain proper coverage over their mouth and nose to eradicate pathogens before they enter their mouth and nose, and also to disinfect the air exhaled from their mouth and nose.

The subject matter disclosed and claimed herein, in one embodiment thereof, comprises a far-UVC radiating protective eyeglasses/eyewear device. The far-UVC radiating protective eyeglass device comprises a frame to wear in front of a user's eyes, a right temple attached to the frame and configured to be placed on the user's right ear, a left temple attached to the frame and configured to be placed on the user's left ear and a plurality of LED wafers positioned on the bottom of the frame and below the lens portion, wherein the LED wafers project far-UVC light in a downward direction at an angle 45-degrees to the eyeglass plane. The far-UVC light forms a virtual shield to disinfect the inhaled air before it reaches the user's mouth and nose and also disinfects exhaled air from the user's mouth and nose. A rechargeable battery is disposed on one of the temples for providing power to the LED wafers and an ON/OFF button is disposed on one of the temples to turn on or off the emission of the far-UVC light. The eyeglass device also comprises a micro-USB charging port for recharging the rechargeable battery.

In this manner, the novel protective eyewear device of the present invention accomplishes all of the forgoing objectives and provides a relatively safe and convenient device which provides protection from pathogens, viruses and bacteria. The present invention eliminates the use of uncomfortable masks and shields by providing an eyewear device that can be worn like conventional eyeglasses, even by ill and asthmatic users. The eyewear device can be used conveniently at any enclosed or public place, and is secure and safe for both the user and other people.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a far-UVC radiating protective eyeglass device. The far-UVC radiating protective eyeglass device comprises a frame configured to be wearable by a user, a right temple attached to the frame and configured to be placed on a user's right ear, a left temple attached to the frame and configured to be placed on a user's left ear, and a plurality of LED wafers positioned on the bottom of the frame and below the lens portion, wherein the LED wafers project far-UVC light in a downward direction at an angle 45-degrees to the eyeglass plane. Thus, the far-UVC light forms a virtual shield to disinfect inhaled air before it reaches a user's mouth and nose, and also disinfects the exhaled air from the user's mouth and nose. A rechargeable battery is disposed on one of the temples for providing power to the LED wafers, and an ON/OFF button is disposed on one of the temples for turning on or off the emission of the far-UVC light. The eyeglass device also comprises a micro-USB charging port for recharging the rechargeable battery.

In yet another embodiment of the present invention, the wavelength of the far-UVC light emitted by the LED wafers is in the range 207-222 nm and the emitted far-UVC light is non-harmful for human and other animals.

In yet another embodiment of the present invention, the ON/OFF button is disposed on the right temple and the battery and micro-USB charging port are disposed on the left temple of the eyeglass device.

In yet another embodiment, the rechargeable battery and the LED wafers are connected through a wired circuitry and the ON/OFF button and the LED wafers are also connected through the same wired circuitry or a different wired circuitry.

In yet another embodiment of the present invention, a novel protective eyewear device is disclosed. The novel protective eyewear device includes a left lens frame, a right lens frame, a right temple, a left temple, a first bracket connected to the bottom of the right lens frame through a pair of first connectors, a second bracket connected to the bottom of the left lens frame through a pair of second connectors, wherein both the first bracket and the second bracket comprise a plurality of LED wafers for emitting far-UVC lights in the range 207-222 nm in a downward direction at an angle of substantially 45-degrees to the brackets for creating a shield for pathogens in both exhaled and inhaled air. A rechargeable battery is disposed on one of the temples for providing power to the LED wafers, and an ON/OFF button disposed on one of the temples for turning on or off the emission of the far-UVC light. The eyeglass device also comprises a micro-USB charging port for recharging the rechargeable battery.

In yet another embodiment of the present invention, the length of the connectors is approximately one inch, and can be integrated or detachably attached to the frames. In yet another embodiment, a method for protecting a user from airborne pathogens using an eyeglass device is described. The method includes providing a far-UVC radiating eyeglass device, worn on the temples before going out in public or coming close to another person, activating the LED wafers using the ON/OFF button disposed on one of the temples of the eyeglass device, emitting and projecting far-UVC lights from the glass frame or via the attached bracket portion that acts as a shield and disinfecting the inhaled air and exhaled air for protection against germs and viruses such as COVID-19, bacteria, etc., wherein the far UVC radiation rays are directed at a 45-degree angle away from the user to create an effective virtual face shield over a user's nose and mouth and finally, turning off the emission of the UV radiations from the UVC LED wafer lights.

In a further embodiment of the present invention, the personal protective eyeglass device of the present invention allows users to use an eyewear/eyeglass device instead of facemasks for effective protection against pathogens. The eyewear device is easy and comfortable to wear and does not limit the breathing of the user. The eyewear is safe for the skin of the user and other people.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the ultraviolet (UV) rays radiating protective eyeglass device of the present invention in accordance with the disclosed architecture;

FIG. 2 illustrates a perspective view showing another potential embodiment of the ultraviolet (UV) rays radiating protective eyeglass device of the present invention in accordance with the disclosed architecture;

FIG. 3 illustrates a flow diagram showing the steps performed by a user using one potential embodiment of the ultraviolet (UV) rays radiating protective eyeglass device of the present invention for protection from airborne pathogens and viruses in accordance with the disclosed architecture;

FIG. 4 illustrates a perspective view of one potential embodiment of the far UVC radiating protective eyeglass device of the present invention in use in accordance with the disclosed architecture; and

FIG. 5 illustrates a planar view showing the orientation of the projection of the far-UVC lights and eyeglass device of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an improved personal protective device that provides a shield to protect users against the spread of germs, viruses and bacteria. There is also a long felt need in the art for an improved personal protective device that is both comfortable to the user and effective in protection from pathogens. Additionally, there is a long felt need in the art for an improved personal protective device that can be used by anyone, including elderly, asthmatic and sick users. Moreover, there is a long felt need in the art for an improved personal protective device that may be in a different form than facemasks and face shields. Further, there is a long felt need in the art for an improved personal protective device that can be used in high-traffic public areas like retail stores, schools, offices, etc. Finally, there is a long felt need in the art for an improved personal protective device that allows users to maintain proper coverage over their noses and mouths to eradicate pathogens and disinfect the air.

The present invention, in one exemplary embodiment, is a novel protective eyewear device. The novel protective eyewear device includes a left lens frame, a right lens frame, a right temple, a left temple, a first bracket connected to the bottom of the right lens frame through a pair of first connectors, a second bracket connected to the bottom of the left lens frame through a pair of second connectors, and wherein both the first bracket and the second bracket comprise a plurality of LED wafers for emitting far-UVC lights in the range 207-222 nm in a downward direction at an angle of substantially 45-degrees to the brackets for creating a shield for pathogens in both exhaled and inhaled air. A rechargeable battery is disposed on one of the temples for providing power to the LED wafers, and an ON/OFF button is disposed on one of the temples for turning on or off the emission of the far-UVC light.

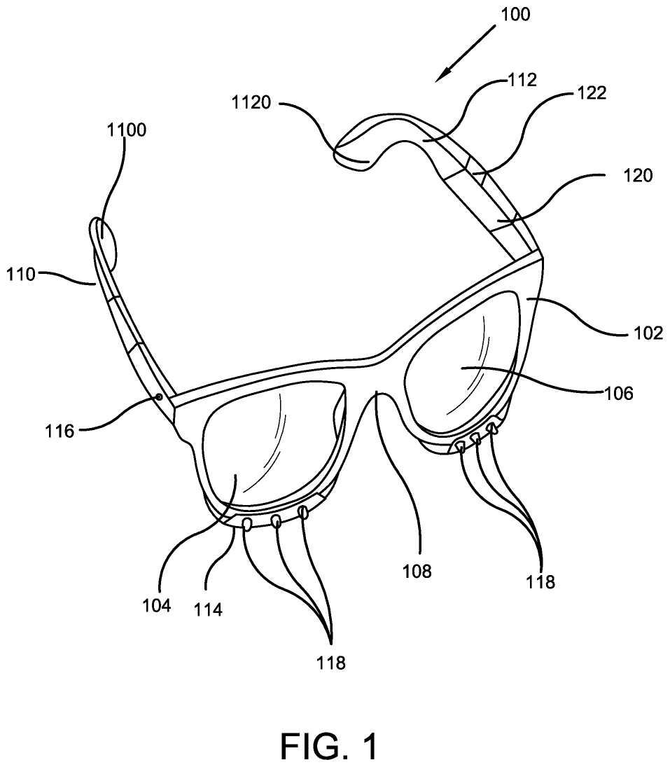

Referring initially to the drawings, FIG. 1 illustrates a perspective view of the ultraviolet (UV) rays radiating protective eyeglass device of the present invention. The UV rays radiating protective eyeglass device 100 of the present invention is in the form of conventional eyeglasses, and is designed to protect the user from the spread of germs and viruses such as COVID-19, bacteria, etc., or any other germs or viruses known in the art. The protective eyeglass device 100 is configured to be used as personal protective equipment and can replace facemasks and face shields, thereby allowing users to breathe easily while also providing adequate protection from airborne pathogens. The protective eyeglass device 100 is ideal for use in places such as high-traffic public areas like retail stores, schools, offices, etc., or other suitable areas as are known in the art for providing protection from contamination due to air borne pathogens and other microbes. More specifically, the eyeglass device 100 comprise a frame 102 adapted to fit around the eyes of a user. The frame 102 includes a right lens portion 104 and a left lens portion 106, designed to receive lenses, and a nose bridge 108 that connects the right lens portion 104 and the left lens portion 108 together. The eyeglass device 100 further comprises a right temple 110 and a left temple 112. The right temple 110 has a right temple tip 1100, and the left temple 112 has a left temple tip 1120 for secure placement of the temples on the ears of a user. A wide variety of lenses are supported by the eyeglass device 100, including but not limited to: standard prescription glasses, non-prescription glasses, sunglasses, 3D glasses, glasses with magnifying lenses, safety glasses, etc., or any other type of suitable glasses as is known in the art.

Both the right arm 110 and the left arm 112 are attached to the frame 102 using hinges or any other mechanical fasteners as used in conventional eyeglasses. Both the right arm 110 and the left arm 112 can be shaped or molded to extend directly from the frame portion 102 and can optionally be attached via mechanical fasteners or other attachment mechanisms as is known in the art.

The frame 102 comprises a plurality of LED wafer UV lights 118 on the rim portion 114 of the frame 102 wherein the rim 114 is disposed at the bottom of the right lens portion 104 and the left lens portion 106. The one or more LED wafer UV lights 118 are designed to generate far UVC radiation projected at approximately a 45-degree angle over the nose and mouth of a user. The far UVC light is projected such that it does not fall on the face, but project outwards from a user's face. The advantage of the far UVC radiation emitted by the LED wafer UV lights 118 is that far-UVC light emits a wavelength in the range 207-222 nm and efficiently inactivates bacteria and other pathogens, without causing harm to the exposed skin of a user and other people. Advantageously, the far-UVC light emitted by the LED wafer UV lights 118 cannot penetrate even the outer layers of human skin or eyes. The far UVC radiation rays generated from the one or more UV LEDs 118 can be used to destroy the outer protein coating of viruses such as SARS-Coronavirus, or SARS-CoV-2 virus, etc., which leads to the inactivation of the viruses. The UV lights 118 emit the far UVC radiation rays at a 45-degree angle over the nose and the mouth of a user, such that the far UVC radiation rays create a shield over the user's nose and mouth to prevent the spread of bacteria and viruses. The far UVC radiation disinfects the inhaled air before it enters the mouth and nose of a user, and disinfects the exhaled air so that other surrounding people remain safe. The far UVC radiation is radiated in a very low dose which is strong enough to kill viruses and pathogens, but does not affect a user.

The right temple 110 includes an ON/OFF toggle button 116 that enables the user to switch on or off the emission of the far UVC lights from the LED wafer 118. The ON/OFF button 116 is coupled to the LED wafer lights 118 through an internal wired circuit within the right temple 110 and the frame 102

The left temple 112 comprises a built-in and removable rechargeable battery 120 for providing power to the LED wafer lights 118. The battery 120 can be a button battery and a minute Li-Ion battery, or any other suitable battery as is known in the art. A micro-USB charging port 122 is provided on the left temple 112 as well. The rechargeable battery 120 can be used to power the one or more UV lights 118 of the eyeglass device 100, and the micro-USB charging port 122 can be used to charge the battery 120 using a charging cord. It should be noted that the battery 120 and the UVC lights 118 are connected through one or more wired circuits within the eyeglass device 100.

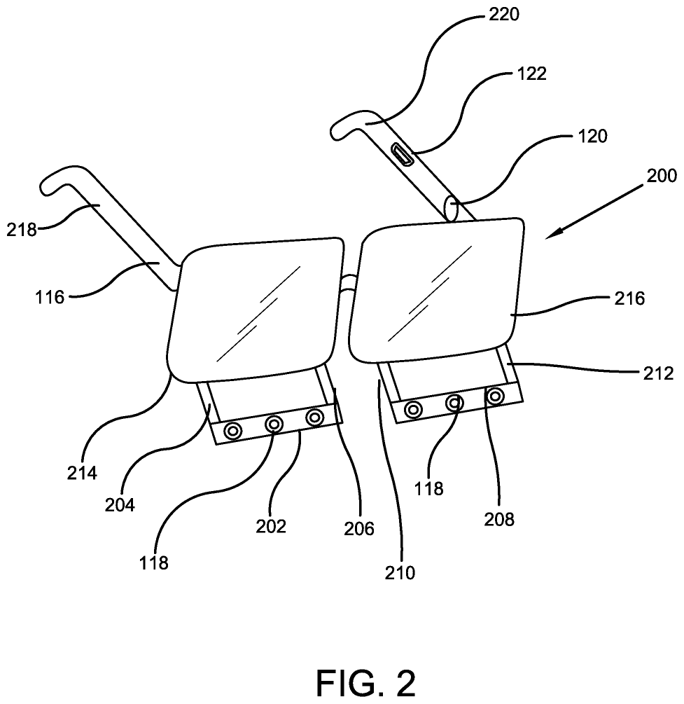

FIG. 2 illustrates a perspective view showing another embodiment of the ultraviolet (UV) rays radiating protective eyeglass device of the present invention. In the present embodiment, the bracket protective eyeglass device 200 comprises UV lights 118 disposed on a right bracket 202 and a left bracket 208. The right bracket 202 is connected to the right lens frame 214 through a pair of connectors 204, 206. The connectors 204, 206 can be integrated to the right lens frame 214, or can be removably-attached to the right lens frame 214. Similarly, the left bracket 208 is connected to the left lens frame 216 through a pair of second connectors 210, 212. The second connectors 210, 212 can be integrated to the left lens frame 216 or can be removably-attached to the left lens frame 216.

The UV lights 118 disposed on both the right bracket 202 and the left bracket 208 emit far UVC radiation having wavelength in the range 207-222 nm that effectively inactivates bacteria and other pathogens without causing harm to exposed skin of a user and other people. The UV lights 118 shine far UVC light over a user's nose and mouth for creating a planar shield for protection against pathogens.

The right temple 218 includes a ON/OFF toggle button 116 that enables the user to switch on or off the emission of far UVC lights from the LED wafer 118 for providing desired protection against germs, viruses and bacteria. The left temple 220 has a built-in and removable rechargeable battery 120 for providing power to the LED wafer lights 118. A micro-USB charging port 122 is provided on the left temple 220 as well. The rechargeable battery 120 powers the one or more UV lights 118 of the eyeglass device 100. The micro-USB charging port 122 can be used to charge the battery 120 using a charging cord.

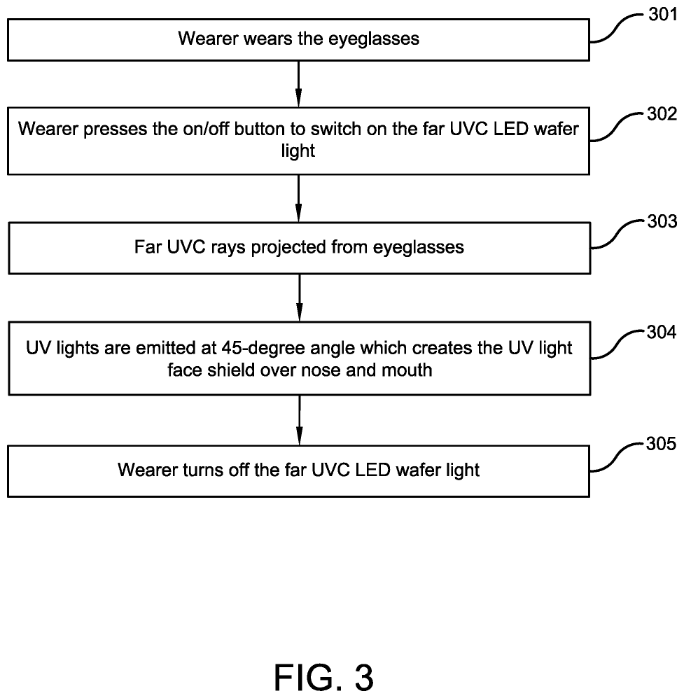

FIG. 3 illustrates a flow diagram showing the steps performed by a user of the ultraviolet (UV) rays radiating protective eyeglass device of the present invention. Initially at 301, a user wears the eyeglass device before going out in public or coming close to another person. The eyeglass device is designed to be used as personal protective equipment in places such as high-traffic public areas like retail stores, schools, offices, etc. Then, at 302, as per a user's wants and/or needs, the user activates the ON/OFF button disposed on the arm of the eyeglass device to switch on the LED wafer far UVC lights. At 303, once the user activates the UV lights, far UVC rays are emitted and projected from the glass frame or via the attached bracket portion. The far UVCs rays act as a shield and disinfect the inhaled air and exhaled air for protection against germs and viruses such as COVID-19, bacteria, etc. Then at 304, the far UVC radiation rays are directed at a 45-degree angle away from the user, to create an effective virtual face shield over a user's nose and mouth. The UV light virtual face shield eliminates germs, viruses or bacteria before they enter the respiratory passages or exit a user's mouth and nose. Finally at 305, the user turns off the emission of the UV radiation from the UVC LED wafer lights.

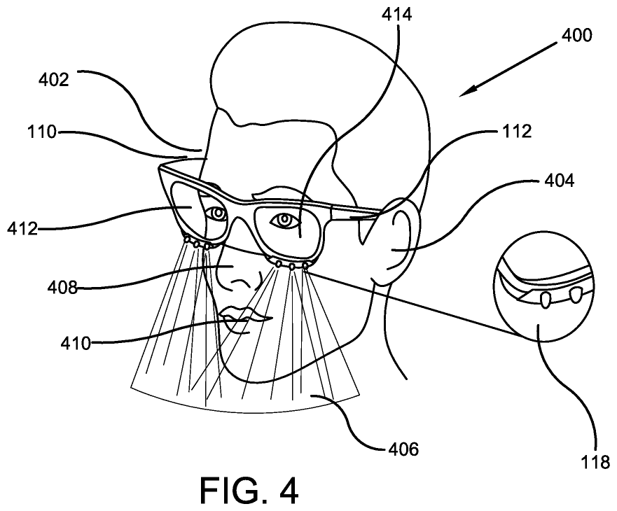

FIG. 4 illustrates a perspective view of the far UVC radiating protective eyeglass device of the present invention in use. As shown, a user 400 wears the protective eyeglass device 100 as conventional eyeglasses by placing the right temple 110 on a right ear 402, and placing the left temple 112 on the left ear 404, such that the right lens 412 and the left lens 414 cover the eyes of the user 400. The UVC LED wafer lights 118 emit and project the far-UVC lights downwards from the eyeglass device 100 to create a virtual protective shield 406 of far-UVC lights in front of the nose 408 and mouth 410 of the user 400. The virtual protective shield 406 of the far-UVC lights disinfects the inhaled air from the external environment before it reaches a user's nose 408 and mouth 410, and disinfects the exhaled air from the nose 408 and mouth 410 of a user 400.

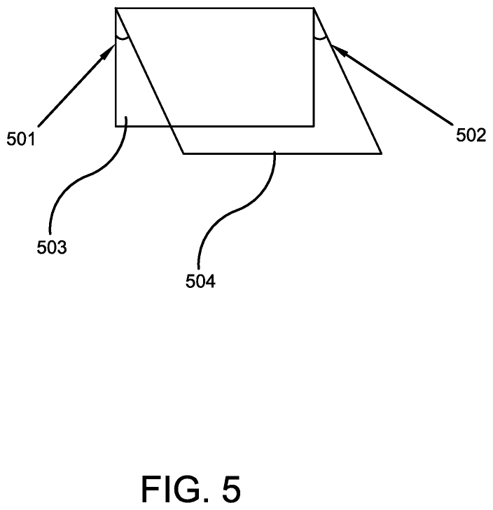

FIG. 5 illustrates a planar view showing the orientation of the projection of the far-UVC lights and eyeglass device. As stated earlier, the far-UVC lights are projected in a plane 504 that is approximately 45-degrees (shown through 501, 502) to the eyeglass device plane 503. The 45-degrees is advantageous as the emitted far-UVC lights does not touch or fall upon the nose and mouth of the user but provide a considerable lateral gap in the range of approximately 6″-8″ from the user's nose and mouth.

The present invention eliminates the use of facemasks and face shields and thus provides a convenient, safe and effective solution for the protection from pathogens. The eyeglass device of the present invention can be manufactured of material such as plastic or light metal, etc., or any other suitable material as is known in the art. The eyeglass device can come in various sizes and colors to meet the needs and/or wants of a user.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “ultraviolet (UV) rays radiating protective eyeglass device”, “far UVC radiating protective eyeglass device”, “protective eyeglass device”, “eyeglass device”, “eyewear device”, “personal protective eyeglass device”, “personal protective eyewear device” and “bracket protective eyeglass device are interchangeable and refer to the ultraviolet (UV) rays radiating protective eyeglass device 100 of the present invention.

Notwithstanding the forgoing, the ultraviolet (UV) rays radiating protective eyeglass device 100 of the present invention can be of any suitable size and configuration as is known in the art without affecting the overall concept of the invention, provided that it accomplishes the above-stated objectives. One of ordinary skill in the art will appreciate that the size, configuration, and material of the ultraviolet (UV) rays radiating protective eyeglass device 100 as shown in FIGS. 1-5 is for illustrative purposes only, and that many other sizes and shapes of the ultraviolet (UV) rays radiating protective eyeglass device 100 are well within the scope of the present disclosure. Although the dimensions of the ultraviolet (UV) rays radiating protective eyeglass device 100 are important design parameters for user convenience, the ultraviolet (UV) rays radiating protective eyeglass device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A protective eyeglass device that protects a user from a spread of germs and viruses, the protective eyeglass device comprising:
a frame comprising a right lens portion, a left lens portion and a nose bridge, wherein the nose bridge connects the rights lens portion and the left lens portion together;
a right temple;
a left temple;
a right arm;
a left arm, wherein the right arm and the left arm are secured to the frame; and
a plurality of LED wafer UV lights positioned on the frame, wherein the plurality of LED wafer UV lights are designed to generate far UVC radiation to efficiently inactivate bacteria and other pathogens without causing harm to an exposed skin of the user.

2. The protective eyeglass device of claim 1, wherein the frame is adapted to fit around eyes of the user.

3. The protective eyeglass device of claim 1, wherein the right lens portion and the left lens portion are configured to receive lenses.

4. The protective eyeglass device of claim 3, wherein the right temple comprises a right temple tip.

5. The protective eyeglass device of claim 4, wherein the left temple comprises a left temple tip.

6. The protective eyeglass device of claim 5, wherein each of the right temple tip and the left temple tip provide for secure placement of the right temple and the left temple on a respective ear of the user.

7. The protective eyeglass device of claim 1, wherein each of the right arm and the left arm are secured to the frame via a hinge.

8. The protective eyeglass device of claim 1, wherein each of the right arm and the left arm are molded to extend directly from the frame.

9. The protective eyeglass device of claim 1, wherein the plurality of LED wafer UV lights are positioned on a rim portion of the frame.

10. The protective eyeglass device of claim 9, wherein the rim portion is disposed at a bottom of each of the right lens portion and the left lens portion.

11. The protective eyeglass device of claim 1, wherein the plurality of LED wafer UV lights are designed to generate far UVC radiation projected at approximately a 45-degree angle over a nose and mouth of the user.

12. The protective eyeglass device of claim 11, wherein the plurality of LED wafer UV lights emits a wavelength in a range of 207-222 nm.

13. The protective eyeglass device of claim 1, wherein the right temple comprises an ON/OFF toggle button to turn on or off the plurality of LED wafer UV lights.

14. The protective eyeglass device of claim 13, wherein the left temple comprises a battery for providing power to the plurality of LED wafer UV lights.

15. The protective eyeglass device of claim 14, wherein the left temple comprises a micro-USB charging port used to charge the battery.

16. A protective eyeglass device that protects a user from a spread of germs and viruses, the protective eyeglass device comprising:
a frame comprising a right lens portion, a left lens portion and nose bridge, wherein the nose bridge connects the rights lens portion and the left lens portion together;
a right temple;
a left temple;
a right arm;
a left arm, wherein the right arm and the left arm are secured to the frame; and
a plurality of LED wafer UV lights positioned on a right bracket and a left bracket secured to the frame, wherein the plurality of LED wafer UV lights are designed to generate far UVC radiation projected at approximately a 45-degree angle over a nose and mouth of the user to efficiently inactivate bacteria and other pathogens without causing harm to an exposed skin of the user, wherein the right temple comprises an ON/OFF toggle button, and further wherein the left temple comprises a battery and a micro-USB charging port used to charge the battery.

17. The protective eyeglass device of claim 16, wherein the right bracket is connected to the right lens portion through a pair of connectors.

18. The protective eyeglass device of claim 17, wherein the left bracket is connected to the left lens portion through a second pair of connectors.

19. A method of using a protective eyeglass device to efficiently inactivate a bacteria and other pathogens without causing harm to an exposed skin of a user, the method comprising the steps of:
wearing the protective eyeglass device before going out in public or coming close to another person;
activating an ON/OFF button disposed on an arm of the protective eyeglass device to switch on a plurality of LED wafer far UVC lights;
emitting and projecting the plurality of LED wafer far UV lights from the protective eyeglass device;
disinfecting an inhaled air and exhaled air;
directing the plurality of LED wafer far UVC lights at a 45-degree angle away from the user to create an effective virtual face shield over a user's nose and mouth; and
turning off the emission of UV radiation from the plurality of LED wafer far UVC lights.

20. The method of claim 19, wherein the plurality of LED wafer far UVC lights are positioned on a frame or an attached bracket portion of the protective eyeglass device.
